(19) United States
(12) Patent Application Publication
(10) Pub. No.: US 2005/0042289 A1
(43) Pub. Date: Feb. 24, 2005

(54) Tablets and methods for modified release of hydrophylic and other active agents

(75) Inventors: James Shunnan Chu, Palo Alto, CA (US); Yisong Yang, Sunnyvale, CA (US); Chunhong Gu, Los Altos, CA (US); Larry Banda, San Leandro, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Yamanouchi Pharma Technologies, Inc., Norman, OK (US)

(21) Appl. No.: 10/835,214

(22) Filed: Apr. 28, 2004

Related U.S. Application Data
(60) Provisional application No. 60/466,842, filed on Apr. 29, 2003.

(51) Int. Cl. A61K 9/26; A61K 9/14

(52) U.S. Cl. 424/469

(57) ABSTRACT

A novel tablet and methods for making the tablet are provided. The tablet comprises at least one particle containing a pharmaceutically active agent and a gel-forming material comprising a first polymer, a second polymer, and a gelation facilitator agent, and has a sustained release profile for the active agent.

### CROSS-REFERENCES TO RELATED APPLICATIONS

This application claims priority to U.S. Patent Application Ser. No. 60/466,842, filed Apr. 29, 2003, the disclosure of which is hereby incorporated by reference in its entirety for all purposes.

### BACKGROUND OF THE INVENTION

A variety of hydrogel-type preparations have been developed to effect the sustained release of orally ingested drugs. For example, Japanese patent application JP-A-62-120315 discloses a preparation obtained by compression-molding a drug, a hydrogel-forming water-soluble polymer and an enteric coating. JP-A-63-215620 discloses a hydrogel-type preparation with a core having a drug and a water-soluble polymer and an outer layer with a water-soluble polymer as a base. JP-B-40-2053 discloses a sustained-release preparation having a mixture of a drug and a high polymer of ethylene oxide and, as an optional component, a hydrophilic substance. However, all of these preparations are designed to release a drug continuously while the administered preparation is still retained in the upper digestive tract, typically in the stomach and small intestine. They were not intended to provide for a release of a drug in the lower digestive tract including the colon, where little water is available.

Hydrophilic gel-forming preparations have been further developed so that they can provide a sustained release of orally ingested drugs throughout the digestive system, including in the lower digestive tract. For example, EP 0 661 045 describes a preparation adapted to absorb water into its core to undergo substantially complete gelation during its stay in the upper digestive tract. As the tablet moves down the digestive system in the form of a gel to the lower digestive tract, the preparation swells and the gelled outer surface of the tablet erodes gradually releasing the drug. This type of oral tablet is capable of providing a sustained release of the drug throughout the digestive tract, including in the colon. More recently, further improvement has been made to gel-forming preparations for sustained release of active agents to address the situations where the active agents are unstable in the gel-forming preparations or may be released in an undesirable manner when previously known methods are used. For example, U.S. Pat. No. 6,419,954 describes a tablet that comprises a gel-forming material and at least one particle containing an active agent and a coating material. The coating material, which is in contact with the active agent, e.g., on or around the active agent, can physically and/or chemically modify the release of the active agent from the tablet. Upon absorbing water in the digestive tract, the gel-forming material forms a matrix for the active agent-containing particles. The presence of the coating material in the tablet, e.g., on the outside of the active agent-containing particle(s), can control the release of the active agent by, for example, slowing or inhibiting the passage of the active agent out of the tablet and into the digestive system.

There exists a continuing need for further improvement in pharmaceutical preparations with a sustained release profile for an extended time period, e.g., of at least 18 hours and preferably at least 24 hours, that would allow the use of a medicine in a once-a-day regime. The present invention satisfies these and other needs.

### BRIEF SUMMARY OF THE INVENTION

One aspect of the present invention relates to a novel tablet that comprises at least one particle containing a pharmaceutically active agent and a gel-forming material, which are blended together. The gel-forming material contains a first polymer, a second polymer, and a gelation facilitator agent.

In some embodiments, the pharmaceutically active agent is in contact with a coating material. Preferably, the coating material is on or around the pharmaceutically active agent, both components of the particle or particles comprising the active agent. In other embodiments, the pharmaceutically active agent is hydrophilic. In yet other embodiments, the gelation facilitator agent has a solubility higher than about 0.1 gram/ml in water at a temperature of about 20° C. In some further embodiments, the tablet has multiple particles comprising the pharmaceutically active agent. Preferably, the gel-forming material forms a matrix for the multiple particles.

In some embodiments, the first polymer is a polyethylene oxide polymer, which, preferably, has an average molecular weight of at least about 4×10⁶ Daltons. In other embodiments, the gelation facilitator agent is polyethylene glycol, which preferably is PEG400, PEG800, PEG1000, PEG1200, PEG1500, PEG2000, PEG4000, PEG6000, PEG8000, PEG10000, or PEG20000, and more preferably PEG6000 or PEG8000. In yet other embodiments, the second polymer consists of one or more polysaccharides selected from the group consisting of locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic. Preferably, the second polymer is xanthan gum. In some further embodiments, the ratio of the first polymer to the gelation facilitator agent is between about 1:0.03 to about 1:40, preferably between about 1:0.1 to about 1:20, more preferably between 1:0.2 to about 1:10, and most preferably between 4:3 to about 3:4 by weight. In some embodiments, the tablet provides a sustained release of the pharmaceutically active agent for at least about 12 hours and preferably for at least about 18 hours. In other embodiments, the pharmaceutically active agent has a solubility of about 0.8 gram/ml in water at a temperature of about 25° C.

A second aspect of the present invention relates to a method for producing a tablet. The method comprises the first step of producing a mixture of at least one particle containing a pharmaceutically active agent and a gel-forming material, which are blended together. The gel-forming material contains a first polymer, a second polymer, and a gelation facilitator agent. The second step of the method is compressing the mixture from the first step to produce the tablet.

In some embodiments, the pharmaceutically active agent is in contact with a coating material. Preferably, the coating material is on or around the pharmaceutically active agent, both components of the particle or particles comprising the active agent. In other embodiments, the pharmaceutically active agent is hydrophilic. In yet other embodiments, the gelation facilitator agent has a solubility higher than about 0.1 gram/mil in water at a temperature of about 20° C. In some further embodiments, the tablet has multiple particles comprising the pharmaceutically active agent. Preferably, the gel-forming material forms a matrix for the multiple particles.

In some embodiments, the first polymer is a polyethylene oxide polymer, which, preferably, has an average molecular weight of at least about 4×10⁶ Daltons. In other embodiments, the gelation facilitator agent is polyethylene glycol, which is preferably PEG400, PEG800, PEG1000, PEG1200, PEG1500, PEG2000, PEG4000, PEG6000, PEG8000, PEG10000, or PEG20000, and more preferably PEG6000 or PEG8000. In yet other embodiments, the second polymer consists of one or more polysaccharides selected from the group consisting of locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic. Preferably, the second polymer is xanthan gum. In some further embodiments, the ratio of the first polymer to the gelation facilitator agent is between about 1:0.03 to about 1:40, preferably between about 1:0.1 to about 1:20, more preferably between 1:0.2 to about 1:10, and most preferably between 4:3 to about 3:4 by weight. In some embodiments, the tablet provides a sustained release of the pharmaceutically active agent for at least about 12 hours and preferably for at least about 18 hours. In other embodiments, the pharmaceutically active agent has a solubility of about 0.8 gram/ml in water at a temperature of about 25° C.

A third aspect of the present invention relates to a method for generating a predetermined sustained release profiled of a pharmaceutically active agent. The pharmaceutically active agent is present in at least one particle that is blended with a gel-forming material, which contains a first polymer, a second polymer, and a gelation facilitator agent. The claimed method achieves distinct release profiles by adapting different weight percentages of the first polymer, the second polymer, and the gelation facilitator agent in the gel-forming material.

In some embodiments, the pharmaceutically active agent is in contact with a coating material. In other embodiments, the first polymer is a polyethylene oxide polymer, which, preferably, has an average molecular weight of at least about 4×10⁶ Daltons. In yet other embodiments, the gelation facilitator agent is polyethylene glycol, which is preferably PEG400, PEG800, PEG1000, PEG1200, PEG1500, PEG2000, PEG4000, PEG6000, PEG8000, PEG10000, or PEG20000, and more preferably PEG6000 or PEG8000. In some further embodiments, the second polymer consists of one or more polysaccharides selected from the group consisting of locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic. Preferably, the second polymer is xanthan gum.

### BRIEF DESCRIPTION OF THE DRAWINGS

FIG. 1 shows the dissolution profile of a tertiary polymer matrix system prepared in Example 1.

FIG. 2 shows the dissolution profile of a tertiary polymer matrix system prepared in Example 6.

### DEFINITIONS

The term “sustained release,” when used to describe the manner an active ingredient is released from a tablet, refers to the fact that the tablet is capable of releasing the active agent to the body for a prolonged period of time, e.g., for at least about 18 hours, and preferably for at least about 24 hours. Preferably, a sustained release tablet releases the active agent from the tablet gradually into the body. For example, a sustained release tablet that is designed to release of the active agent for about 18-24 hours preferably has the following dissolution specification using the dissolution test method described in Example 6A: no more than 40% of the active agent (e.g., by weight) released in 1 hour, about 70-85% of the active agent released in 12 hours, and no less than about 80% of the active agent released at 24 hours. In another example, a sustained release tablet is designed to release the active agent at a nearly linear zero order rate (typically when the active agent dissolution is measured up to 70% of the active agent release).

The “size” of active granules or pellets, or coated beads or coated particles refers to the average dimension and can be measured by either laser diffraction sizer analysis or mechanical siever such as Ro-Tap.

Unless specified otherwise, a range of “molecular weight” of a polymer (e.g., a polyethylene oxide polymer or a polysaccharide) or a gelation facilitator agent (e.g., a polyethylene glycol) described below is a weighted average molecular weight (measured by gel permeation chromatography).

The term “cps” or “centipoise” is a unit of viscosity=m Pascal second. The viscosity is measured by Broolfield or other viscometers. See, e.g., Wang (1998) Clin. Hemorheol. Microcirc. 19:25-31; Wang (1994) J. Biochem. Biophys. Methods 28:251-61; Cooke (1988) J. Clin. Pathol. 41:1213-1216.

Tablet “hardness” is physical strength measurement of the tablet. The resistance of a tablet to chipping, abrasion, or breakage under conditions of storage, transportation and handling before usage depends on its hardness, or “crushing strength.” The tablet “crushing” or “tensile” strength is defined as the force required to break a tablet by compression in the radial direction. It is typically measured using one of the many commonly available tablet hardness testers. For example, “Stokes” and “Monsanto” hardness testers measure the force required to break the tablet when the force generated by a coil spring is applied diametrically to the tablet. A “Strong-Cobb” hardness tester also measures the diametrically applied force required to break a tablet, the force applied by an air pump forcing a plunger against the tablet placed on an anvil. Electrically operated hardness testers, such as the Schleuniger apparatus (also known as a “Heberlein”) can be used. See also, TS-50N, Okada Seiko Co., Japan; Bi (1996) Chem. Pharm. Bull. (Tokyo) 44:2121-2127. The tablet hardness can be represented by various units, including in the units of kilopounds (“kp”).

The “gelation index” or “percent gelation” as used herein represents the percentage of the portion of the tablet which has undergone gelation. The method of calculating the gelation index is not particularly limited but the following calculation method may be mentioned as an example.

Using The Pharmacopeia of Japan XII (referred to “JP” hereinafter) Disintegration Test Fluid 2, a gelation test can be carried out by JP Dissolution Test Method 2 (paddle method) at a paddle speed of 25 rpm. The test tablet is moistened for a predetermined time. The test tablets are then taken out at predetermined intervals, the gel layer is removed and the diameter (D obs) of the portion not forming a gel can be measured. From this D obs value, the gelation index (G) can be calculated (see Equation 1 below).

$$Gelation Index ( G , \% ) = ( 1 - \frac{{( D obs )}^{3}}{{( D ini )}^{3}} ) \times 100$$

D obs: The diameter of the portion not gelled after initiation of test

D ini: The diameter of the preparation before initiation of test

As an alternative to measuring the diameter of the tablet, other parameters, such as volume, weight or thickness, of the tablet can be measured to calculate gelation index.

A “first polymer” as used herein refers to a composition that comprises a polymer such as a polyethylene oxide polymer. A “second polymer” as used herein refers to a composition that comprises one or more polymers. Polysaccharides are preferred polymers components of the “second polymer,” which can comprise, e.g., locust bean gum, xanthan gum, tragacnth, xylan, arabinogalactan, agar, gellan gum, scleroglucan, guar gum, apricot gum (Prunus armeniaca, L.), alginate, carrageenan, acacia gum, dragon gum, hog gum, talha, dextran, gum Arabic, and combinations thereof.

### DETAILED DESCRIPTION OF THE INVENTION

In one aspect, the invention provides a modified tablet comprising a gel-forming material and at least one particle comprising an active agent, wherein the particle is formulated to modify release of the active agent from the tablet. The gel-forming material, comprising a first polymer, a second polymer, and a gelation facilitator, forms a matrix (i.e., a gel-forming matrix) for the active agent-containing particles in the modified tablet. In some embodiments, the particle comprises a pharmaceutically active agent and a coating material on or around the active agent, wherein the coating material further modifies the release of the active agent from the tablet. The gel-forming matrix, and optionally the coating material, can provide any desired active agent release profile. For example, embodiments of the invention can provide a sustained release of a pharmaceutically active ingredient, even one with high water solubility, from the tablet for at least 18 hours, preferably for at least 24 hours. Depending on the ultimate use of the tablets, these tablets typically comprise components that are physiologically or pharmacologically acceptable.

The gel-forming material of the present invention can comprise: (1) a first polymer; (2) a second polymer; and (3) a gelation facilitator agent. The first polymer is water insoluble and contributes to forming a network of materials within the matrix which can swell upon absorbing water. The second polymer comprises at least one polymer, or it can comprise a mixture of two or more polymers. Polysaccharides are the most preferred type of polymer(s) in the second polymer. Also water insoluble, the second polymer interacts with the first polymer to form a matrix that is more resistant to erosion in the digestive tract and can further retard the release of the active agent from the tablet. The gelation facilitator agent is a hydrophilic base that draws water into the core of the gel-forming matrix of the tablet, thereby allowing a substantially complete gelation of the entire tablet before the tablet reaches the large intestine. Preferably, the gelation facilitator agent has a solubility higher than about 0.1 gram/ml in water at a temperature of about 20° C. Different forms and/or types of the polymers and the gelation facilitator agent can be used to modify the gelation rate and/or erosion rate of the gel matrix. They can be selected to provide a controlled release pattern of the active agent-containing particles. Other additives can be incorporated to further modify the gelation and/or release pattern of the active agent.

The particle is formulated to further modify the release of the active agent (in particular the hydrophilic agent) from the tablet. Typically, the particle comprises an active agent and an optional coating material on, and preferably around, the active agent. The active agent can be in any suitable form. In certain embodiments, the active agent can be in the form of a crystal, a granule, or a pellet. These active agent forms may facilitate certain coating processes of the active agents. Moreover, the particle can comprise a single active agent crystal (or granule or pellets) or can comprise a plurality of active agent crystals (or granules or pellets).

In another aspect, the tablets are designed to have pulsatile or delayed onset release profiles. This can be achieved by designing, e.g., a multilayered tablet. Different layers of the multilayered tablet can have different active agents, different amounts of active agents, different forms of active agents, different amounts or kinds of coating materials, different amounts or kinds of gel-forming materials, etc.

In a further aspect, the invention provides a method for generating a predetermined profile of sustained release of an active ingredient from a tablet of the present invention by choosing proper weight percentages of the first polymer, the second polymer, and the gelation facilitator agent in the gel-forming material. An maximal delaying effect in releasing an active agent can be achieved by including a coating material around the particle(s).

I. Active Agents and Pelletization/Granulation Development

Any suitable active agent can be incorporated into the embodiments of the present invention. Preferably, the active agent is a drug. However, the active agent is not necessarily limited to a drug, but can be a nutritional additive (e.g., vitamin), a placebo, or a reagent (e.g., a diagnostic reagent, a radioimaging reagent, or a magnetic imaging reagent).

In one embodiment, the active agent is hydrophilic and has a water solubility of at least about 10 mg/ml at a temperature of about 25° C. Optionally, a hydrophilic active agent has a water solubility of at least about 50 mg/ml, at least about 100 mg/ml, at least about 200 mg/ml, at least about 300 mg/ml, at least about 400 mg/ml, at least about 500 mg/ml, at least about 600 mg/ml, at least about 700 mg/ml, at least about 800 mg/ml, at least about 900 mg/ml, at least about 1,000 mg/ml, at least about 1,200 mg/ml, or at least about 1,500 mg/ml at a temperature of about 25° C. Examples of a hydrophilic active agent includes, cevimeline HCl, pseudoephedrin HCl, pyrilamine maleate, phenmetrazine HCl, hyoscyamine sulfate, edophonium HCl, doxylamine succinate, hydroxyzine HCl, fluphenazune HCl, niacinamide, metprolol HCL, and the like.

In certain embodiments, the active ingredient of the tablet is (+/−)-cis-2-methylspiro [1,3-oxathiolane-5,3′-quinuclidine] hydrochloride, hemihydrate; also known as SNI-2011, cevimeline hydrochloride, AF102B, SND-5008, and FKS-508. SNI-2011 is a rigid analogue of acetylcholine. See, e.g., Iga (1998) Jpn. J. Pharmacol. 78:373-380; Iwabuchi (1994) Arch. Int. Pharmacodyn. Ther. 328(3):315-25. It is distributed by, e.g., Snow Brand Milk Product Co., Shinjuku-Ku, Tokyo, Japan. Quinuclidine salt derivative analogues of SNI-2011 can also be used. SNI-2011 and analogues are highly water soluble drugs, having a water solubility of about 1,400 mg/ml at 25° C. Cevimeline hydrochloride has a water solubility of 766 mg/ml at 25° C. They specifically bind to specific muscarinic receptors in various exocrine glands. They have demonstrated beneficial effects on xerostomia and keratoconjunctivitis sicca in patients with Sjogren's syndrome, see, e.g., Iwabuchi (1994) Gen. Pharmacol. 25:123-129. Preferably, the daily dose of these drugs that can achieve 18-24 hour extended release is incorporated into the tablet. Typically, about 1 mg to about 500 mg, optionally about 10 mg to about 200 mg, optionally 50 mg to about 100 mg, or optionally about 75 mg to about 90 mg of the drug, e.g., Cevimeline hydrochloride, is incorporated into the tablet. This dosage could accommodate once a day dosing of the drug.

In another embodiment, an active agent can be non-hydrophilic (e.g., a hydrophobic) or can have a water solubility of less than, e.g., 30 mg/ml or 20 mg/ml at a temperature of about 25° C.

In yet another embodiment, an active agent is a drug that is unstable if it is in contact with water or a gel-forming matrix for a prolonged period of time (e.g., sensitive to moisture or oxidation). These active agents may benefit by having a physical and/or chemical barrier (e.g., a coating material). Examples of unstable drugs include antibiotic drugs such as efrotomycin, milbemycins, tylosin derivatives, avermectins, ivermectin, mocimycin, goldinomycin, and the like.

Any other suitable active agents can be included in the embodiments of the invention. For example, the pharmaceutically active agents include, but not limited to, e.g., anti-inflammatory, antipyretic, anticonvulsant and/or analgesic agents such as indomethacin, diclofenac, diclofenac Na, codeine, ibuprofen, phenylbutazone, oxyphenbutazone, mepirizol, aspirin, ethenzamide, acetaminophen, aminopyrine, phenacetin, scopolamine butylbromide, morphine, etomidoline, pentazocine, fenoprofen calcium, etc; tuberculostats such as isoniazid, ethambutol hydrochloride, etc.; cardiocirculatory system drugs such as isosorbide dinitrate, nitroglycerin, nifedipine, barnidipine hydrochloride, nicardipine hydrochloride, dipyridamole, arinone, indenolol hydrochloride, hydralazine hydrochloride, methyldopa, furosemide, spironolactone, guanethidine nitrate, reserpine, amosulalol hydrochloride, amitriptyline hydrochloride, neomapride, haloperidol, moperone hydrochloride, perphenazine, diazepam, lorazepam, chlordiazepoxide, etc.; antihistaminic agents such as chlorpheniramine maleate, diphenhydramine hydrochloride, etc.; vitamins such as thiamine nitrate, tocopherol acetate, cycothiamine, pyridoxal phosphate, cobamamide, ascorbic acid, nicotinamide, etc.; antigout agents such as allopurinol, colchicine, probenecid, etc.; hypnotic sedatives such as amobarbital, bromovalerylurea, midazolam, chloral hydrate, etc.; antineoplastic agents such as fluorouracil, carmofur, aclarubicin hydrochloride, cyclophosphamide, thiotepa, etc.; anticongestants such as phenylpropanolamine, ephedrine, etc.; antidiabetics such as acetohexamide, insulin, tolbutamide, etc.; diuretics such as hydrochlorothiazide, polythiazide, triamterene, etc.; bronchodilators such as aminophylline, formoterol fumarate, theophylline, etc; antitussives such as codeine phosphate, noscapine, dimemorfan phosphate, dextromethorphan, etc; antiarrhythmic agents such as quinidine nitrate, digitoxin, propafenone hydrochloride, procainamide, etc.; surface anesthetics such as ethyl aminobenzoate, lidocaine, dibucaine hydrochloride, etc.; antiepileptics such as phenytoin, ethosuximide, primidone, etc.; synthetic adrenocortical steroids such as hydrocortisone, prednisolone, triamcinolone, betamethasone, etc.; digestive system drugs such as famotidine, ranitidine hydrochloride, cimetidine, sucralfate, sulpiride, teprenone, plaunotol, etc.; central nervous system drugs such as indeloxazine, idebenone, tiapride hydrochloride, bifemelane hydrochloride, calcium hopantenante, etc.; hyperlipemia treating agents such as pravastatin sodium etc.; and antibiotics such as ampicillin phthalidyl hydrochloride, cefotetan, josamycin and so on. Typical drugs among the above drugs are nicardipine hydrochloride, SNI-2011, nifedipine, ditilazem hydrochloride, phenylpropanolamine hydrochloride, indomethacin, potassium hydrochloride, diazepamtheophylline, verapamil, morphine, and the like.

In some embodiments of the invention, the pharmaceutically active agent may also contain a selective cyclooxygenase-2 inhibitor. For example, these cyclooxygenase-2 inhibitors may include substituted pyrazolyl benzenesulfonamides such as 4-[5-(4-methylphenyl)-3-(trifluoromethyl)-1H-pyrazol-1-yl]benzenesulfonamide, or celecoxib, and 4-[5-(3-fluoro-4-methoxyphenyl)-3-difluoromethyl)-1H-pyrazol-1-yl]benzenesulfonamide, or deracoxib (U.S. Pat. No. 5,760,068); substituted isoxazolyl benzenesulfonamides such as 4-[5-methyl-3-phenylisoxazol-4-yl]benzenesulfonamide, or valdecoxib (U.S. Pat. No. 5,633,272); substituted (methylsulfonyl)phenyl furanones such as 3-phenyl-4-[4-(methylsulfonyl)phenyl]-5H-furan-2-one, or refecoxib (U.S. Pat. No. 5,474,995), 3-(1-cyclopropylmethoxy)-5,5-dimethyl-4-[4-(methylsulfonyl)phenyl]-5H-furan-2-one, and 3-(1-cyclopropylethoxy)-5,5-dimethyl-4-[4-(methylsulfonyl)phenyl]-5H-furan-2-one (U.S. Pat. No. No. 5,981,576); substituted pyridines such as 5-chloro-3-(4-methylsulfonyl)phenyl-2-(2-methyl-5-pyridinyl)pyridine, or etoricoxib (U.S. Pat. No. 5,861,419); 2-(3,5-difluorophenyl)-3-[4-(methylsulfonyl)phenyl]-2-cyclopenten-1-one (EP 0 863 134); benzopyrans such as (S)-6,8-dichloro-2-(trifluoromethyl)-2H-1-benzopyran-3-carboxylic acid (U.S. Pat. No. 6,034,256); and substituted pyridazinones such as 2-(3,4-difluorophenyl)-4-(3-hydroxy-3-methyl-1-butoxy)-5-[4-(methylsulfonyl)phenyl]-3-(2H)-pyridazinone (WO 00/24719).

In the embodiments of the invention, the active agent can be in any suitable form. For example, it can be in the form of a particle, powder, a crystal, or a granule (i.e., an aggregate of smaller units of active agent, also referred to as a pellet). Depending on whether coating is necessary on the particle(s) containing the active agents and/or the methods used to coat the active agents to produce a particle, the active agents may be used as purchased (in the form of a powder or a crystal) or may be processed to form active agent granules or pellets. For example, if active agent granules or pellets are spray coated with a coating material to form coated active agent-containing particles, the active agents are preferably granulated or pelletized to improve the chemical or physical characteristics of the active agents for coating processes. For certain coating processes, it may be preferable that active agents are in the form of a granule or a pellet that has, e.g., relatively high density and hardness, and relatively low brittleness and surface area.

An active agent can be pelletized or granulated using any suitable methods known in the art. Pelletization or granulation is commonly defined as a size-enlargement process in which small particles are gathered into larger, permanent aggregates in which the original particles can still be identified. Prior to granulation, a binder can be added to the active agent to improve the granulation process. Examples of a suitable binder includes, hydroxypropyl cellulose (HPC), a mixture of polyethylene oxide and polyethylene glycol, acacia, carbomer, carboxymethylcellulose sodium, ethylcellulose, dextrin, gelatin, guar gum, hydroxyethyl cellulose, hydroxypropyl methylcellulose, maltodextrin, povidone, pregelatinized starch, zein, starch, and the like. Other additives can be added during granulation. These include, e.g., sweeteners, flavors, color agents, antioxidants, etc.

Optionally, water or other solvents can be added to aid the granulation process. The amount of water or solvent added depends on, e.g., the selection of a granulation process, and is readily determinable by those of skill in the art. Water or other solvent may be added at any suitable time point during the granulation process. For example, a binder may be mixed with a solvent (e.g., water) to form a binder solution, and then the binder solution can be sprayed onto active agents. Alternatively, if a binder solution is too viscous to be uniformly sprayed onto active agents, it may be desirable to blend the binder with the active agent first and then spray water or other solvent to produce uniform pattern of active agent granules or pellets.

Any suitable granulation methods can be used to produce particles comprising an active agent. By definition, granulation is any process of size enlargement whereby small particles are gathered together into larger, permanent aggregates to render them into a free-flowing state. For example, either wet granulation or dry granulation methods can be used.

Dry granulation refers to the granulation of a formulation without the use of heat and solvent. Dry granulation technology generally includes slugging or roll compaction. Slugging consists of dry-blending a formulation and compressing the formulation into a large tablet or slugs on a compressing machine. The resulting tablets or slugs are milled to yield the granules. Roller compaction is similar to slugging, but in roller compaction, a roller compactor is used instead of the tableting machines. See, e.g., Handbook of Pharmaceutical Granulation Technology, D. M. Parikh, eds., Marcel-Dekker, Inc. pages 102-103 (1997). Dry granulation technique is useful in certain instances, e.g., when the active agent is sensitive to heat or solvent.

Alternatively, wet granulation can be used. In wet granulation, solvents and binders are typically added to a formulation to provide larger aggregates of granules. The temperature during granulation can be set at any suitable point, generally not exceeding the melting point of any components of the formulation. Typically, the mixture is granulated at a temperature of about 35° C. to about 65° C. for about 20 to 90 minutes. Then the granules are typically air dried for a suitable duration (e.g., one or more hours).

Preferably, the active agents are granulated with high shear mixer granulation (“HSG”) or fluid-bed granulation (“FBG”). Both of these granulation processes provide enlarged granules or pellets but differ in the apparatuses used and the mechanism of the process operation. In HSG, blending and wet massing is accomplished by high mechanical agitation by an impeller and a chopper. Mixing, densification, and agglomeration of wetted materials are achieved through shearing and compaction forces exerted by the impeller. The primary function of the chopper is to cut lumps into smaller fragments and aid the distribution of the liquid binder. The liquid binder is either poured into the bowl or sprayed onto the powder to achieve a more homogeneous liquid distribution.

On the other hand, fluidization is the operation by which fine solids are transformed into a fluid-like state through contact with a gas. At certain gas velocities, the fluid will support the particles, giving them freedom of mobility without entrainment. Such a fluidized bed resembles a vigorously boiling fluid, with solid particles undergoing extremely turbulent motion, which increases with gas velocity. Fluidized bed granulation is then a process by which granules are produced in FB by spraying a binder solution onto a fluidized powder bed to form larger granules. The binder solution can be sprayed from, e.g., a spray gun positioned at any suitable manner (e.g., top or bottom). The spray position and the rate of spray may depend on the nature of the active agent and the binder used, and are readily determinable by those skilled in the art.

These granulation techniques can be performed using commercially available apparatuses. For example, the HSG can be performed using Aeromatic-Field GP1/SP General Processor. Depending on the properties of the active agent, the HSG process is preferred. For example, when Cevimeline HCl was granulated using either HSG or FBG processes, it was found that the HSG process generated the Cevimeline HCl granules or particles of higher density than the FBG process.

Optionally, granulated active agents can be milled. Milling can be performed using any commercially available apparatuses (e.g., Comil equipped with a 0.039 inch screen). The mesh size for the screen can be selected depending on the size of the active agent granule or pellet desired. Typically, the mesh size can range from 0.331 inch screen (mesh 20) to 0.006 inch screen (mesh 100). The milling process aids in providing relatively uniform active agent granules. After the granulated active agents are milled, they may be further dried (e.g., in the air) if desired.

Typically, the mean size of the active granule or pellet can range from about 50 μm to about 3 mm, optionally about 100 μm to about 2 mm, about 300 μm to about 1 mm. Typically, the bulk density or the tap density of the active agent granules or pellets range from about 0.1 g/ml to about 1.5 g/ml, optionally about 0.3 to about 0.8 g/ml, optionally about 0.4 g/ml to about 0.6 g/ml. Bulk density is measured based on USP method (see US Pharmacopoeia, edition XXIV, pages 1913-1914, testing method <616>, incorporated herein by reference).

II. Preparation of Particles Comprising an Active Agent and an Optional Coating Material

Active agents (as purchased or as pelletized) are processed to form a particle with an optional coating material. When present, the coating material is in contact with the active agents. Any suitable coating material can be selected to modify the release of the active agent from the tablet in any suitable manner. For example, the particle comprises an active agent and a coating material on or around the active agent. Advantageously, this physical/chemical modification of the active agent provides improved sustained or controlled release of active agents from the tablet.

Any suitable coating material may be used with the embodiments of the invention. For example, the coating material can be a natural polymer, a semi-synthetic polymer, or a synthetic polymer. These include, but are not limited to, chitosan, methylcellulose, ethylcellulose, hydroxyethylcellulose, hydroxypropylcellulose hydroxyethylmethylcellulose, hydroxypropylmethylcellulose, cellulose acetate membrane, cellulose acetate butyrate, cellulose acetate propionate, cellulose acetate phthalate, hydroxypropylmethylcellulose phthalate, polyacrylic acid, polyvinyl acetate, poly(vinylacetate phthalate), poly(vinyl alcohol), poly(vinyl pyrrolidone), poly(lactic acid), poly(glycolic acid), poly(lactic/glycolic acid), poly(dimethyl silicone), poly(hydroxyethyl methacrylate), poly(ethylene/vinyl acetate), poly(ethylene/vinyl alcohol), polyamides, polyesters, polyurethanes, polyureas, or a mixture thereof.

In one embodiment, the coating material is a less hydrophilic than the active agent so that the coating material inhibits the diffusion of the hydrophilic active agent through the tablet. Typically, a water insoluble or hydrophobic coating material can be used. Examples of a hydrophobic coating material include ethyl cellulose, polymethacrylic polymers, polyvinylacetate, cellulose acetate polymers, etc.

In another embodiment, the coating material is flexible so that it is flexible to withstand the compression pressure during the production of compressed tablets. A tablet is generally compressed to a hardness of at least about 2 kp, typically between about 2 kp to about 10 kp. Accordingly, the coating material preferably comprises sufficient flexibility, plasticity, or elasticity so that it does not deform (e.g., crack or break) during the tablet compression of at least about 2 kp. A plasticizer may be included in the coating material to increase the flexibility of the coating material. Examples of a plasticizer include benzyl benzoate, chlorobutanol, dibutyl sebacate, diethyl phthalate, clycerin, mineral oil, polyethylene glycol, sorbitol, triacetin, triethyl citrate, etc. Optionally, a stabilizer, such as acacia, bentonite, cyclodextrins, glyceryl monostearate, propylene glycol, white or yellow wax, Xanthan gum, etc., can be added to a coating material.

Some of these coating materials are in the form of an aqueous polymeric dispersion and are commercially available. For example, these include Surelease™ (ethyl cellulose), Eudragit™ RS/NE (polyacrylic polymers), and Kollicoat™ SR (polyvinylacetate), etc. Typically, these commercially available coating materials include a plasticizer and/or a stabilizer.

Depending on the selection of a coating method, a single active agent crystal or granule or a plurality of active agent crystals or granules can be coated to form a single particle. For example, the particle can comprise a single active agent crystal or granule and coating material on and around the active agent. These particles can be produced by, e.g., spraying a coating material on an individual active agent crystal or granule. As another example, the particle can comprise a plurality of active agents (powders, crystals, or granules) and coating material on or around the active agents. Particles can be produced by, e.g., applying the coating material on the active agent powder, crystals or pellets and then chopping (and optionally milling) the coated mixture into particles. The particles comprising the active agent and the coating materials will be interchangeably referred to as beads or coated beads or coated particles.

Any suitable coating methods can be used to produce particles comprising an active agent and a coating material on or around the active agent. For example, either a type A or a type B coating or encapsulation process can be used.

Type A processes include simple or complex coacervation, interfacial polymerization in liquid media, in-liquid drying, thermal and ionic gelation in liquid media, or desolvation in liquid media techniques. In one embodiment of a type A processes, a coacervation process can be used. In both simple and complex coacervation process, the water phase is utilized as the continuous phase (see, e.g., Ertan (1997) J. Microencapsul. 14:379-388; Tuncel (1996) Pharmazie 51:168-171). In addition, water soluble polymers can be used in the coacervation process. Typical coating or microencapsulating polymers include, e.g., polyamides, polyesters, polyurethanes, and the polyureas and the like.

In another embodiment, the interfacial polymerization process can be used. In this process, two polymeric monomer suspensions are used—a discontinuous phase comprising particles comprising active agents to be encapsulated and a continuous phase for coating films. The two monomers react at the interface between the core and the continuous phase, causing polymerization under controlled process conditions.

In yet another embodiment, a phase separation technique can be used (see, e.g., Mandal (1998) Drug Dev. Ind. Pharm. 24:623-629). In this technique, a polymer of discontinuous phase is generally phased out or desolvated from the solvent continuous phase. This polymer of discontinuous phase will deposit around a reservoir such as a liquid droplet or a solid particle as a polymer wall. The polymer can be deposited either by temperature differential, by the introduction of a second polymer, or by the evaporation of the solvent.

Type B processes utilize spray drying, spray chilling, fluidized bed coating, spray drying, pan coating, and spray coating, electrostatic deposition, centrifugal extrusion, spinning disk or rotational suspension separation, polymerization at liquid-gas or solid-gas interface, pressure extrusion or spraying into solvent extraction bath techniques. Pan coating can be utilized for multiple layer coatings of the tablet (see, e.g., Heinamaki (1997) Pharm. Dev. Techno. 2:357-364). Fluid bed coating, referred to as Wurster coating, and spray drying techniques can also be utilized for coating the particle comprising an active agent (see, e.g., Jono (1999) Chem. Pharm. Bull. (Tokyo) 47:54-63; Miyamoto (1997) Chem. Pharm. Bull. (Tokyo) 45:2043-2050).

When coating of particles is necessary, the selection of a suitable coating process depends on the physical and chemical characteristics of the active agent, the coating material, etc. and is determinable by those skilled in the art. For instance, when a spray system is used to coat particles, it may be desirable to use a coating material that does not have a high tack in its formula which could lead to clogging of the nozzle of the spray system.

Any suitable amount of coating material can be applied on the active agent as long as the coating provides sufficient diffusion or protective barrier for the active agent. Typically, about 10% to about 150 % of the coating is applied to the active agent granule, wherein the % coating means % ratio (w/w) of the amount of coating polymer used to the amount of active agent granule (and a binder and other materials in the granule). Preferably, about 30% to about 80% of coating is applied. More preferably, about 40% to about 60% of coating is applied.

Depending on the amount of coating material applied, the mean size of the particles comprising an active agent and a coating material can range, e.g., from about 50 μm to about 5 mm, optionally about 100 μm to about 3 mm, about 300 μm to about 2 mm. Typically, the bulk density or the tap density of these particles is slightly higher than uncoated active agent granules or pellets. For example, the bulk density or the tap density of the particles can range from about 0.1 g/ml to about 5 g/ml, optionally about 0.3 to about 3 g/ml, optionally about 0.5 g/ml to about 1.0 g/ml. Preferably, the particles are round, even, and smooth. Also preferably, the particles are relatively strong and yet flexible.

Besides controlling active agent release pattern, physical modification of the active agent using encapsulation, granulation, and/or polymer coating techniques have many advantages such as less inter- intra-individual variation, a very reduced influence of gastric emptying and intestinal transit time, reduced influence of pH, viscosity and consequently of food and of the position of the body as well as gastroresistance and taste-masking.

III. Gel-Forming Material and Combining Gel-Forming Material and Particles Comprising an Active Agent

The particles comprising an active agent, and optionally a coating material, are then blended with a gel-forming material. The final blend is then compressed into tablets without damaging the particles. The gel-forming material, which forms a matrix for the particles, comprises a first polymer, a second polymer, and a gelation facilitator agent. The first polymer provides a structural basis for the matrix of the tablet to swell upon absorbing water. The second polymer interacts with the first polymer and serves as an additional structural component for the matrix to reduce the rate of matrix erosion in the digestive tract and thus further retard the release of the active agent. The gelation facilitator agent is a hydrophilic base that draws water into the core of the gel-forming matrix, thereby allowing substantial gelation of the entire tablet. By incorporating the gelation facilitator agent, the gel-forming matrix absorbs water to undergo substantially complete gelation during its stay in the upper digestive tract and moves into the lower digestive tract undergoing constant erosion, continuously releasing the particles comprising the active agent from the tablet.

The first and second polymers in the gel-forming matrix have certain physical characteristics, including viscosity in the gelled state, which permit the tablet to retain its shape to certain extent during its travel down to the lower digestive tract, including the colon, withstanding the contractile forces of the digestive tract associated with the digestion of food. The properties of the first and second polymers depend on their molecular weight, viscosity, etc. The first polymer used in the present invention typically has an average molecular weight ranging from about 0.5×10⁶ Daltons to 10×10⁶ Daltons, more typically ranging from 1×10⁶ Daltons to 8×10⁶ Daltons. Preferably, the first polymer component in the gel-forming material has an average molecular weight of at least about 1×10⁶ Daltons and has a viscosity of at least about 1,000 cps in a 1% water solution at a temperature of about 25° C. (i.e., if 1% by weight of PEO is added to water, the aqueous solution containing PEO has a viscosity of at least about 1,000 cps). More preferably, the first polymer has an average molecular weight of at least about 2×10⁶ Daltons, even more preferably between about 5×10⁶ Daltons to about 10×10⁶ Daltons. Preferably, the second polymer has an average molecular weight in the range of about 5×10³ to 5×10⁷ Daltons and has a viscosity of at least about 1,000-15,000 cps in a 1% water solution at a temperature of about 25° C. More preferably, the second polymer has an average molecular weight in the range of about 5×10⁴ to 1×10⁷ Daltons, and even more preferably between about 5×1 Daltons to about 5×10⁶ Daltons.

Suitable to serve as the first polymers are polyethylene oxide (PEO) [e.g., Polyox WRS-303 (average mol. wt.: 7×10⁶; viscosity: 7500-10000 cps, 1% in H₂O, 25° C.), Polyox WSR Coagulant (average mol. wt.: 5×10⁶; viscosity: 5500-7500 cps, under the same condition above), Polyox WSR-301 (average mol. wt.: 4×10⁶ viscosity: 1650-5500 cps, under the same condition above), Polyox WSR-N-60K (average mol. wt.: 2×10⁶; viscosity: 2000-4000 cps, 2% in H₂O, 25° C.), all of which are trade names of Union Carbide Co.]; hydroxypropylmethylcellulose (HPMC) [e.g., Metolose 90SH10000 (viscosity: 4100-5600 cps., 1% in H₂O, 20° C.), Metolose 90SH50000 (viscosity: 2900-3900 cps, under the same condition above), Metolose 90SH30000 (viscosity: 25000-35000 cps, 2% in H₂O, 20° C.), all of which are trade names of Shin-Etsu Chemicals Co.]; sodium carboxymethylcellulose (CMC-Na) [e.g., Sanlose F-150MC (average mol. wt.: 2×10⁵, viscosity: 1200-1800 cps, 1% in H₂O, 25° C.), Sanlose F-1000MC (average mol. wt.: 42×10⁴; viscosity: 8000-12000 cps, under the same condition above), Sanlose F-300MC (average mol. wt.: 3×10⁵; viscosity: 2500-3000 cps, under the same condition above), all of which are trade names of Nippon Seishi Co., Ltd.]; hydroxyethylcellulose (HEC) [e.g., HEC Daicel SE850 (average mol. wt.: 148×10⁴; viscosity: 2400-3000 cps, 1% in H₂O, 25° C.), HEC Daicel SE900 (average mol. wt.: 156×10⁴; viscosity: 4000-5000 cps, under the same condition above), all of which are trade names of Daicel Chemical Industries]; carbonxyvinyl polymers [e.g., Carbopol 940 (average mol. wt.: ca. 25×10⁵; B.F. Goodrich Chemical Co.) and so on.

In a preferred embodiment, a PEO is used as a first polymer as part of the gel-forming material. Where a continuous release of the drug over a long time is desired, a first polymer having a higher molecular weight, preferably an average molecular weight of more than 4×10⁶ Daltons, or a higher viscosity, preferably a viscosity of more than 3000 cps at a concentration of 1% in water at 25° C., is preferable.

Preferred polymers to serve as components of a second polymer have similar physical properties, such as high molecular weight and high viscosity. In general, one or more polysaccharides are preferred polymers as second polymers in the gel-forming material. They include, but are not limited to, locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic. Some molecular weight and viscosity information is as follows: Xanthan Gum (average molecular weight: 2×10⁶; viscosity: 1200-1600 cps, 1% in H₂O, 25° C.), tragacanth (average molecular weight: 8.4×10⁵; viscosity: 100-4000 cps, 1% in H₂O, 25° C.), Guar gum (average molecular weight: 2.2×10⁵; viscosity: 2000-3500 cps, 1% in H₂O, 25° C.), Acacia gum (average molecular weight: 2.4-5.8×10⁵; viscosity: 100 cps, 30% in H₂O, 25° C.).

In order to ensure that a drug is steadily released during a time period of at least 18 hours and preferably 24 hours following administration, it is desired that a portion of the preparation having undergone gelation still retains some degree of structural integrity at such time. To provide a tablet having such properties, although it depends on the volume of the preparation, the kind of polymer and the properties and amount of the active agent and of the gelation facilitating agent (for ensuring a penetration of water into the preparation core), it is generally preferable that the formulation contains about 1 to about 85 weight % (preferably about 5 to about 60 weight %, and more preferably about 5 to about 40 weight %) of the polymer (e.g., based upon the preparation weighing less than 600 mg). One preparation contains not less than 20 mg per preparation and preferably not less than 30 mg per preparation of the polymer. If the amount of this polymer is less than the above-mentioned level, the preparation may not tolerate erosion in the digestive tract and may not achieve a sustained release of the active agent.

The gelation facilitator agent allows water to penetrate into the core of the tablet. The higher the solubility of the gelation facilitator agent in water, the more effective it is in allowing into the core of the tablet. The gelation facilitator agent as used in one embodiment as one of the components of the gel-forming material can be at least one excipient having solubility higher than 0.1 g/ml in water at room temperature (e.g., 20° C). Preferably, the amount of water required to dissolve 1 gram (g) of the gelation facilitator agent is not more than 5 ml, and more preferably not more than 4 ml at room temperature (e.g., 20° C).

Examples of such gelation facilitator agent includes highly hydrophilic polymers such as different molecular weight polyethylene glycol (PEG), e.g., polyethylene glycols (PEG), e.g. PEG400, PEG800, PEG1000, PEG1200, PEG1500, PEG2000, PEG4000, PEG6000, PEG8000, PEG10000, PEG20000, and the like (produced by, e.g., Nippon Oils and Fats Co.), or mixtures thereof. Other highly hydrophilic polymers that can be used as gelation facilitator agents include polyvinylpyrrolidone (PVP; e.g. PVP K30™, PVP K90™ from BASF), hydroxyethylcellulose, hydroxypropylcellulose, and the like; sugar alcohols such as D-sorbitol, xylitol, etc.; sugars such as sucrose, anhydrous maltose, D-fructose, dextran (e.g. dextran 40), glucose, etc.; surfactants such as polyoxyethylene-hydrogenated castor oil (HCO; e.g Cremophor RH40™ produced by BASF, HCO-40™ and HCO-60™ produced by Nikko Chemicals Co.), polyoxyethylene-polyoxypropylene glycol (e.g. Pluronic F68™ produced by Ashai Denka Kogyo K.K.), polyoxyethylene-sorbitan fatty acid ester (Tween; e.g. Tween 80 produced by Knato Kagaku K.K.), etc.; salts such as sodium chloride, magnesium chloride, etc.; organic acids such as citric acid, tartaric acid, etc.; amino acids as glycine, β-alanine, lysine hydrochloride, etc.; and amino sugars such as meglumine.

In a preferred embodiment, a polyethylene glycol (PEG) is used as a gelation facilitator agent. Typically, a PEG used in the embodiments of the invention has an average molecular weight between about 4×10² Daltons and about 2×10⁴ Daltons. Preferably, a PEG having an average molecular weight between about 400 Daltons to about 1500 Daltons is used.

The proportion of such a gelation facilitator agent (to the first polymer, such as a PEO polymer) depends on the characteristics of the drug (solubility, therapeutic efficacy, etc.) and content of the drug, solubility of the gelation facilitator agent itself, characteristics of the PEO polymer used, the patient's condition at the time of administration and other factors. For administration to human patients, however, the proportion may preferably be a sufficient level to achieve a substantially complete gelation in about 2 to 5 hours after administration. The proportion of the gelation facilitator agent is, therefore, generally about 1% to about 90% by weight, preferably about 5% to about 60% by weight, more preferably about 5% to about 40% by weight, based on the total weight of the preparation. When the content of the gelation facilitator agent is too small, the necessary gelation into the core of the preparation does not proceed so that the release of the drug in a delayed fashion becomes insufficient. On the other hand, when the content of the gelation facilitator agent is excessive, the gelation proceeds in a shorter time but the resulting gel becomes so fragile that the release of the active agent is too fast, thus failing to ensure a sufficient sustained release. Preferably, the amount of the gelation facilitator agent in the tablet is such that the tablet can achieve at least about 70%, preferably at least about 80% gelation or gelation index after two hours according to the gelation test provided in the definition section above (see, also, EP 0 661 045 A1, incorporated herein by reference). The percentage of PEO in the gel-forming material is generally about 1-85%, preferably about 5-60%, and more preferably about 5-40%; PEG is generally about 1-85%, preferably about 5-60%, and more preferably about 5-40%; Xanthan gum is generally about 10-90%, preferably about 20-70%, and more preferably about 40-60%.

The release property of the particles comprising an active agent can be manipulated by adjusting the first polymer to gelation facilitator agent ratios in the formulation of the tablets. For example, ratios of the polymer to gelation facilitator agent can be between about 1:0.03 to about 1:40 by weight; about 1:0.1 to about 1:20 by weight; about 1:0.2 to about 1:10 by weight. Typically, as the proportion of the polymer increases in the formulation, a slower rate of active agent release can be observed. With a specific combination of a polymer (i.e., PEO) and a gelation facilitator agent (i.e., PEG), however, it was observed that there is no further retardation of active agent release when the PEO content increases beyond a PEG:PEO ratio of about 3:4 to about 4:3. The preferred level of the second polymer in the gel-forming material is generally about 10-90%, preferably about 20-70%, and more preferably about 40-60% for retarded release of an active agent.

In certain embodiments, polymers such as hydroxypropylmethylcellulose (HPMC), sodium carboxymethylcellulose (CMC-Na), hydroxyethylcellulose (HEC), carboxyvinyl polymers, and the like can be added to the tablet formulation to adjust and thus program the release pattern of the active agent.

If desired, the preparation of the present invention may include appropriate amounts of other pharmaceutically acceptable additives such as vehicles (e.g., lactose, mannitol, potato starch, wheat starch, rice starch, corn starch, and crystalline cellulose), binders (e.g., hydroxypropylmethylcellulose, hydroxypropylcellulose, methylcellulose, and gum arabic), swelling agents (e.g., carboxymethylcellulose and carboxy-methylcellulose calcium), lubricants (e.g., stearic acid, calcium stearate, magnesium stearate, talc, magnesium meta-silicate aluminate, calcium hydrogen phosphate, and anhydrous calcium hydrogen phosphate), fluidizers (e.g., hydrous silica, light anhydrous silicic acid, and dried aluminum hydroxide gel), colorants (e.g., yellow iron sesquioxide and iron sesquioxide), surfactants (e.g., sodium lauryl sulfate, sucrose fatty acid ester), coating agents (e.g., zein, hydroxypropylmethylcellulose, and hydroxypropylcellulose), aromas (e.g., f-menthol, peppermint oil, and fennel oil), and preservatives (e.g., sodium sorbate, potassium sorbate, methyl p-benzoate, and ethyl-benzoate).

Any suitable methods can be used to mix the formulation comprising the particles (comprising an active agent and an optional coating material on the active agent) and the gel-forming materials (e.g., the first polymer, the second polymer, and the gelation facilitator agent). In one embodiment, the particles and the gel-forming materials are combined, and the mixture may be directly compressed into a tablet. Typically, one or more vehicles or additives may be added to the mixture to improve flow and compressible characteristics. These additives include, for example, lubricants, such as magnesium stearate, zinc stearate, stearic acid, talc, and the like; flavors; and sweeteners. Direct compression has advantages, such as reducing cost, time, operational pace, and machinery; preventing active agent-excipient interaction; and less instability of active agent. Direct blending or slugging can also eliminate the possible pollution by organic solvent.

In another embodiment, some of the formulation components may be partially granulated prior to compression or all of the formulation components may be granulated prior to compression. For example, some or all components of the gel-forming material can be granulated prior to mixing the active agent-containing particles. In another embodiment, the first polymer (e.g., PEO) and the second polymer (e.g., a polysaccharide) of the gel-forming material can be granulated prior to mixing with the gelation facilitator agent and/or with the particles. In yet another embodiment, the gelation facilitator agent of the gel-forming material can be granulated prior to mixing with the first and second polymers and/or the particles. In still yet another embodiment, the particles comprising an active agent can be granulated together with the gel-forming material (e.g., the first polymer, the second polymer, the gelation facilitator agent, or all three). If any of the gel-forming material is granulated first, preferably, the granules of the gel-forming material are soft or flexible enough not to damage the active agent-containing particles during compression.

Any suitable granulation methods can be used to mix the formulation. In one embodiment, a wet granulation process can be used to mix one or more components of the formulation. For example, high shear granulation or fluid-bed granulation processes can be used. Any suitable commercially available granulation apparatuses can be used in these processes. In another embodiment, a dry granulation process can be used to mix one or more components of the formulation. For example, slugging or roller compaction can be used.

After the granulation of one or more components of the formulation, optionally, granulated formulation can be milled. Milling can be performed using any suitable commercially available apparatus (e.g., Comil equipped with a 0.039 inch screen). The mesh size for the screen can be selected depending on the size of the granules desired. After the granulated active agents are milled, they may be further dried (e.g., in the air) if desired.

After preparing the formulation as described above, the formulation is compressed into a tablet form. This tablet shaping can be done by any suitable means, with or without compressive force. For example, compression of the formulation after the granulation step can be accomplished using any tablet press, provided that the tablet composition is adequately lubricated. The level of lubricant in the formulation is typically in the range of 0.5-2.0%, with magnesium stearate which is most commonly used as a lubricant. Many alternative means to effectuate this step are available, and the invention is not limited by the use of any particular apparatus. The compression step can be carried out using a rotary type tablet press. The rotary type tableting machine has a rotary board with multiple through-holes, or dies, for forming tablets. The formulation is inserted into the die and is subsequently press-molded.

The diameter and shape of the tablet depends on the molds, dies, and punches selected for the shaping or compression of the granulation composition. Tablets can be discoid, oval, oblong, round, cylindrical, triangular, and the like. The tablets may be scored to facilitate breaking. The top or lower surface can be embossed or debossed with a symbol or letters.

The compression force can be selected based on the type/model of press, what physical properties are desired for the tablets product (e.g., desired, hardness, friability, etc.), the desired tablet appearance and size, and the like. Typically, the compression force applied is such that the compressed tablets have a hardness of at least about 2 kp. These tablets generally provide sufficient hardness and strength to be packaged, shipped or handled by the user. If desired, a higher compression force can be applied to the tablet to increase the tablet hardness. However, the compression force is preferably selected so that it does not deform (e.g., crack or break) active agent-containing particles within the tablet. Preferably, the compression force applied is such that the compressed tablet has a hardness of less than about 10 kp. In certain embodiments, it may be preferred to compress a tablet to a hardness of about between about 3 kp to about 7 kp, optionally between about 3 kp to about 5 kp, or about 3 kp.

Typically, the final tablet will have a weight of about 100 mg to about 2000 mg, more typically about 200 mg to about 1000 mg, or about 400 mg to about 700 mg.

If desired, other modifications can be incorporated into embodiments of the invention. For example, modification of drug release through the tablet matrix of the present invention can also be achieved by any known technique, such as, e.g., application of various coatings, e.g., ion exchange complexes with, e.g., Amberlite IRP-69. The tablets of the invention can also include or be coadministered with GI motility-reducing drugs. The active agent can also be modified to generate a prodrug by chemical modification of a biologically active compound which will liberate the active compound in vivo by enzymatic or hydrolytic cleavage; etc. Additional layers of coating can act as barriers for diffusion to provide additional means to control rate and timing of drug release.

### IV. Generation of a Predetermined Sustained Release Profile of a Pharmaceutically Active Agent

In another aspect, the present invention provides methods for generating a predetermined sustained release profile of a pharmaceutically active agent. As described in the previous sections, the tablets of the invention comprise at least one pharmaceutically active agent-containing particle, which may contain an additional coating material, and a gel-forming material, which comprises a first polymer, a second polymer, and a gelation facilitator agent, the profile for the sustained release of the pharmaceutically active agent depends on factors such as the choice of the components of the gel-forming material, their respective proportions, and whether any coating material is included in the particles containing the active agent. Thus, a desired release profile of a pharmaceutically active agent can be achieved by varying the levels of the first polymer, the second polymer, and the gelation facilitator, e.g., the ratios of the first polymer to the gelation facilitator agent by weight. By adding an optional layer of coating on or around the active agent, the sustained release profile of the active agent can be further modified.

A more complex “programmable release profile,” which can comprise multiple stages in releasing active agent(s) with distinct release profile, can be achieved by combining layers of gel-forming material with varying formulations, e.g., with varying percentages of one or more of the three main components of the gel-forming material. In addition, the distribution pattern of the active agent-containing particles blended within the gel-forming material can contribute to the sustained release profile of the active agent from the tablet. When the particles are distributed in the gel-forming material non-randomly (e.g., not evenly), a non-constant, but controlled level of active agent delivery can be achieved, such as, e.g., a pulsatile or delayed onset release profile. The tablets also can be designed and manufactured such that “lag times” of release are incorporated into this scheme. For example, the tablets can be designed to have a delayed onset release of about 2 hours, about 3 hours, about 4 hours, about 5 hours, about 6 hours, or about 7 hours, after the administration.

In certain embodiments, the non-random drug distribution is controlled through a multilayer tablet formulation design and manufacturing process. Active agent distribution in the tablet is designed to be uneven (i.e., non-random). This can be achieved by manufacturing the tablet with multiple layers of formulation, with the layers having differing concentrations and/or types (e.g., modifications, pretreatments) of active agent. For example, alternative layers can have, in addition to varying amounts of active agent, particles comprising the same active agent by different amounts of coating materials or different compositions of coating materials, and the like, or varying amounts of any combination of these alternative forms. The layers can be of varying thickness. Moreover, one tablet can have one, two, three, four, fix, six, seven, eight, nine, ten, or any number of layers, limited only by the desired size of the finished tablet product, the thickness of each layer, the composition of each layer's formulation, the manufacturing process, and the like.

Various “pulsatile release” profiles can be designed by varying the rate at which the tablet dissolves as it passes through the digestive tract. This is accomplished by manufacturing different layers of the multilayered tablet with different kinds or amounts of first polymer (e.g., PEO of varying molecular weights), different ratios of first polymer to gel facilitator, different percentages of second polymer (e.g., one or more polysaccharides) in the gel-forming material, different manufacturing compression forces, and the like. Thus, in addition to having different amounts or different modifications of active agent in each layer, the layers themselves can be pre-programmed to dissolve at different rates (and thus release active agent in different anatomical compartments) as the tablet passes through the digestive tract.

Whether the active agents are distributed randomly or non-randomly, a tablet can comprise one or more types of active agent, and/or one or more types of coating materials. The non-random distribution of active agent can be represented quantitatively by different amounts in different layers or qualitatively by having different forms of active agent in different layers, e.g., as having more coating materials in the particle in the outer layers as compared to the inner layers of the tablet, or, vice versa. In alternative embodiments, the non-random distribution of the active agent in the tablet is concentrated at the core of the tablet or is concentrated at the periphery of the tablet. In another embodiment, the tablet has multiple layers comprising varying amount of active agent or other formulation ingredients. Varying amounts of active agent can be in different layers of the multilayered tablet, e.g., increasing amounts of active agent in the outer layers as compared to the inner layers, or vice versa. Alternatively, different forms of active agent (e.g., encapsulated, granulated, conjugated) can be in different layers. Completely different types of active agents (e.g., drugs) or combinations thereof can be placed into different layers. The layers can be of varying thickness. One tablet can have one, two, three, four, five, six, seven, eight, nine, ten, or any number of layers, limited only by the desired size of the finished tablet product, the thickness of each layer, the composition of each layer's formulation, the manufacturing process, and the like.

The manufacture of the varying layers of a multilayered, pulsatile release tablet can be controlled through the compression coating process. A series of feeding devices equal in number to the number of layers to be designed in the tablet is distributed about a rotary disc (this scheme applies for both the direct compression and granulation processes). In operation, each feeding device emits a defined quantity of material into the female dies as the die travel by the feeding device's output valve. Each feeding device has a compressing device directly downstream, as seen in the direction of movement of the female dies. The compressing devices compress the material admitted into the female dies by the respective feeding devices. The compression causes the various layers of material to adhere to one another. Different amount of compressive force can be used for each layer.

When the desired number of layers has been formed, the resulting multilayered compressed tablet is ejected from the female die. Any appropriate apparatus for forming multilayer tablets can be used to make the pulsatile release tablets of the invention, e.g., powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990.

Different layers of the tablet can contain different amounts or kinds of formulation, including, e.g., PEO, PEG, polysaccharide, and/or active agent compositions. This variation in layers controls the amount and distribution of active agent within the tablet and its eventual release upon ingestion. The multilayered tablet can be further processed in any manner, e.g., by powder layering in coating pans or rotary coaters; dry coating by double compression technique, tablet coating by film coating technique, and the like.

The following examples are offered by way of illustration, not by way of limitation. The contents of all U.S. patents and other references cited in this application are hereby incorporated by reference in the entirety.

### EXAMPLES

### Example 1

A tertiary polymer matrix system was evaluated for purpose of a once-daily controlled release for a highly aqueous soluble drug. The hydrophilic polymers, including polyethylene oxide (PEO), polyethylene glycol (PEG), and xanthan gum was utilized as the main formulation component for this controlled release dosage form. Xanthan gum was selected through the screening of a variety of available polymers with swelling or drug release controlling characteristics. A highly aqueous soluble drug of methylspiro hydrochloride salt (˜800 mg/ml at 25° C.), which performs as cholinergic agonist to increase the functions of the salivary and lacrimal glands, was used as the model drug. The formula is set forth in Table 1.

TABLE 1
Formula of Tertiary Matrix System

| Function | Ingredients | % |
|---|---|---|
| Active | Methylspiro HCl | 22.3 |
| Polymer | PEO | 16.4 |
| Gelation Facilitator | PEG | 22.0 |
| Polymer | Xanthan Gum | 38.4 |
| Lubricant | Magnesium Stearate | 1.0 |

Direct blending was applied for all the ingredients and compression was conducted subsequently.

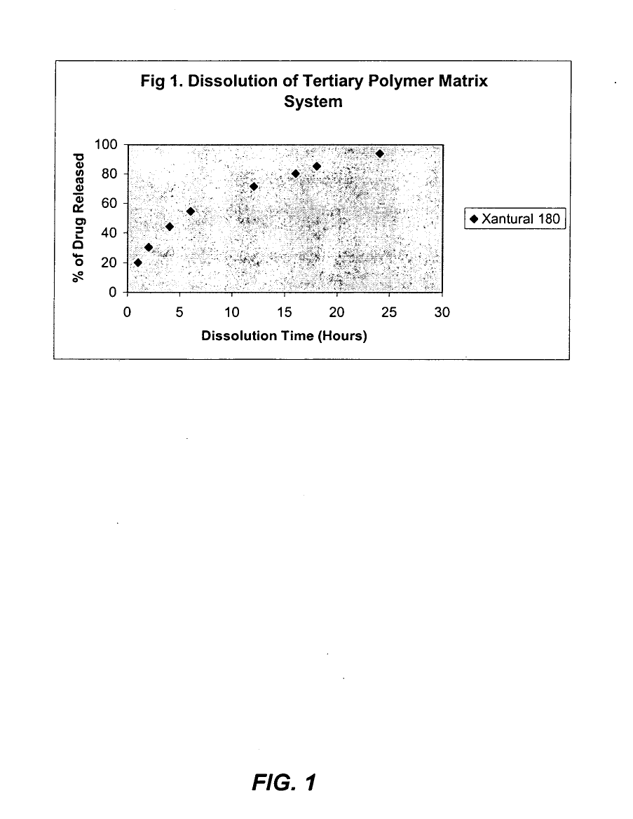

Drug-excipient compatibility and in-vitro dissolution studies were conducted to examine the stability of the additional polymer component. The in-vitro release study from matrix tablets was conducted with USP dissolution apparatus II at 75 rpm in 900 ml 37° C. deionized water. Samples were analyzed by HPLC system using UV detection at 210 nm. The percent of drug released was assessed at various time intervals and the drug release profile can be found in FIG. 1 and in Table 2 below. A near zero order release of active was observed with up to 18 hours release time.

TABLE 2
Percent of drug released over time

| Dissolution Time (Hours) | % of Drug Released |
|---|---|
| 1 | 20.1 |
| 2 | 30.3 |
| 4 | 44.4 |
| 6 | 54.7 |
| 12 | 71.5 |
| 16 | 80.3 |
| 18 | 85.3 |
| 24 | 93.9 |

The drug release kinetics and mechanism were further investigated by applying the simple power law expression Mₜ/M∞=ktⁿ, where Mt/M∞ is the fraction of drug released, k is the kinetic constant and n describes the drug release mechanism. The calculated n values, were 0.43<n<0.85 which are indicative of anomalous transport kinetics, including synergic effects of drug diffusion within the polymer matrix and polymer swelling phenomenon.

### EXAMPLE 2

In this example, the same ingredients were used as in Example 1. Various levels of xanthan gum from 25% up to 75% were examined, with the active content and tablet weight remaining the same as in Example 1. The same direct blending was employed as in Example 1. The data suggests that polymer level is a critical factor in the performance of controlled-release tablets.

### Example 3

In this example, the use of different particle sizes of xanthan gum, XANTURAL 75 and XANTURAL 180 was investigated with the same formula and direct blending method as used in Example 1. The XANTURAL 180 used is a standard grade with particle size around 80 mesh, while XANTURAL 75 has a much finer particle size, around 200 mesh, which allows better dispersion throughout the tablets and rapid hydration once introduced into water. The data suggests that particle size of polymer had less of an effect on the drug release rate.

### Example 4

A similar formula was used in this example as in Example 1. However a different formulation process was applied. The active agent was first coated with Surelease using a Wurster coating process (Glatt granulator with Wurster insert) to produce coated active pellets. Then the coated active pellets were blended with the rest of the ingredients as in Example 1.

### Example 5

A similar formula was used in this example as in Example 1. However, a different formulation process was performed. The active agent was first coated with Kollicoat SR 30D using a Wurster coating process (Glatt granulator with Wurster insert) to produce coated active pellets. The coated active pellets were blended with the rest of the ingredients as in Example 1.

### Example 6

A similar formula is used in this example as in Example 1. However a different formulation process was followed. The active agent was first coated with Surelease using a Wurster coating process (Glatt granulator with Wurster insert) to produce coated active pellets. The coated active pellets along with rest of excipients, PEO, PEG and xanthan gum were sprayed with water to produce active granules with a top spray granulation process.

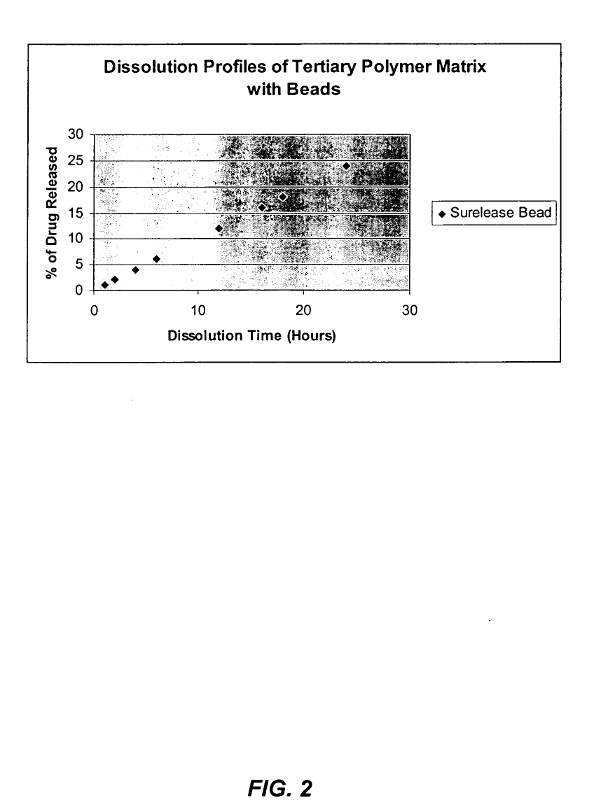

The in-vitro release study from the matrix tablets was conducted with a USP dissolution apparatus II at 75 rpm in 900 ml 37° C. deionized water. The samples were analyzed by a HPLC system using UV detection at 210 nm. The drug release profile of this example can be found in FIG. 2 and in Table 3 shown below. A near zero order release of active was observed with up to 30 hours release time.

TABLE 3
Percent of drug released over time

| Dissolution Time (Hours) | % of Drug Released |
|---|---|
| 1 | 3.4 |
| 2 | 10 |
| 4 | 20.5 |
| 6 | 29.2 |
| 12 | 45.4 |
| 16 | 53.2 |
| 18 | 58.8 |
| 24 | 70.4 |

### Example 7

A similar formula was used in this example as in Example 1. However, a different formulation process was used. The active was first coated with Kollicoat SR 30D using a Wurster coating process (Glatt granulator with Wurster insert) to produce coated active pellets. The coated active pellets along with the rest of excipients, PEO, PEG and xanthan gum were sprayed with water to produce active granules with top spray granulation process.

### Example 8

A similar formula is used in this example as in Example 1. However, a different formulation process was utilized. The core tablets were produced in the same way as in Example 1. The core tablets were then coated with Surelease using pan-coating process.

### Example 9

A similar formula was used in this example as in Example 1. However a different formulation process was utilized. The core tablets were produced in the same way as in Example 1. The core tablets were then coated with Kollicoat SR 30D using a pan-coating process.

### Example 10

A similar formula was used in this example as in Example 1. However, a different formulation process was taken. The active was first coated with Surelease using a Wurster coating process (Glatt granulator with Wurster insert) to produce coated active pellets. The rest of the excipients, PEO, PEG and xanthan gum were made into granules by a roller compactor dry granulation process. The coated active pellets along with excipients dry granules were blended together to provide the final blend for compression.

### EXAMPLE 11

A similar formula was used in this example as in Example 1. However, a different formulation process was taken. The active was first coated with Kollicoat SR 30D using a Wurster coating process (Glatt granulator with Wurster insert) to produce coated active pellets. The rest of the excipients, PEO, PEG and xanthan gum were made into granules by a roller compactor dry granulation process. The coated active pellets along with excipients dry granules was blended together to provide a final blend for compression.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A tablet comprising:
a. at least one particle comprising a pharmaceutically active agent; and
b. a gel-forming material comprising:
i) a first polymer;
ii) a second polymer; and
iii) a gelation facilitator agent,
wherein the at least one particle is blended with the gel-forming material.

2. The tablet of claim 1, wherein the pharmaceutically active agent is in contact with a coating material.

3. The tablet of claim 1, wherein the pharmaceutically active agent is hydrophilic.

4. The tablet of claim 1, wherein the gelation facilitator agent has a solubility higher than about 0.1 gram/ml in water at a temperature of about 20° C.

5. The tablet of claim 2, wherein the at least one particle comprises the pharmaceutically active agent and the coating material on or around the pharmaceutically active agent.

6. The tablet of claim 1, wherein the at least one particle is a plurality of particles.

7. The tablet of claim 6, wherein the tablet comprises the plurality of particles and wherein the gel-forming material forms a matrix for the plurality of particles.

8. The tablet of claim 1, wherein the first polymer is a polyethylene oxide polymer.

9. The tablet of claim 8, wherein the polyethylene oxide polymer has an average molecular weight of at least about 4×10⁶ Daltons.

10. The tablet of claim 1, wherein the gelation facilitator agent is polyethylene glycol.

11. The tablet of claim 10, wherein the polyethylene glycol is a member selected from the group consisting of PEG400, PEG800, PEG1000, PEG1200, PEG1500, PEG2000, PEG4000, PEG6000, PEG8000, PEG10000, and PEG20000.

12. The tablet of claim 11, wherein the polyethylene glycol is PEG6000 or PEG8000.

13. The tablet of claim 1, wherein the second polymer consists of one or more polysaccharides selected from the group consisting of locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic.

14. The tablet of claim 13, wherein the second polymer consists of xanthan gum.

15. The tablet of claim 1, wherein the ratio of the first polymer to the gelation facilitator agent is between about 1:0.03 to about 1:40 by weight.

16. The tablet of claim 15, wherein the ratio of the first polymer to the gelation facilitator agent is between about 1:0.1 to about 1:20 by weight.

17. The tablet of claim 16, wherein the ratio of the first polymer to the gelation facilitator agent is between about 1:0.2 to about 1:10 by weight.

18. The tablet of claim 17, wherein the ratio of the first polymer to the gelation facilitator agent is between about 4:3 to about 3:4 by weight.

19. The tablet of claim 1, wherein the tablet provides a sustained release of the pharmaceutically active agent for at least about 12 hours.

20. The tablet of claim 19, wherein the tablet provides a sustained release of the pharmaceutically active agent for at least about 18 hours.

21. The tablet of claim 1, wherein the pharmaceutically active agent has a solubility of about 0.8 gram/ml in water at a temperature of about 25° C.

22. A method for producing a tablet, said method comprising:
(1) producing a mixture comprising:
a. at least one particle comprising a pharmaceutically active agent; and
b. a gel-forming material comprising:
i) a first polymer;
ii) a second polymer; and
iii) a gelation facilitator agent,
wherein the at least one particle is blended with the gel-forming material; and
(2) compressing the mixture to produce the tablet.

23. The method of claim 22, wherein the pharmaceutically active agent is in contact with a coating material.

24. The method of claim 22, wherein the pharmaceutically active agent is hydrophilic.

25. The method of claim 22, wherein the gelation facilitator agent has a solubility higher than about 0.1 gram/ml in water at a temperature of about 20° C.

26. The method of claim 23, wherein the at least one particle comprises the pharmaceutically active agent and the coating material on or around the pharmaceutically active agent.

27. The method of claim 22, wherein the at least one particle is a plurality of particles.

28. The method of claim 27, wherein the tablet comprises the plurality of particles and wherein the gel-forming material forms a matrix for the plurality of particles.

29. The method of claim 22, wherein the first polymer is a polyethylene oxide polymer.

30. The method of claim 29, wherein the polyethylene oxide polymer has an average molecular weight of at least about 4×10⁶ Daltons.

31. The method of claim 22, wherein the gelation facilitator agent is polyethylene glycol.

32. The method of claim 31, wherein the polyethylene glycol is a member selected from the group consisting of PEG400, PEG800, PEG1000, PEG1200, PEG1500, PEG2000, PEG4000, PEG6000, PEG8000, PEG10000, and PEG20000.

33. The method of claim 32, wherein the polyethylene glycol is PEG6000 or PEG8000.

34. The method of claim 22, wherein the second polymer consists of one or more polysaccharides selected from the group consisting of locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic.

35. The method of claim 34, wherein the second polymer consists of xanthan gum.

36. The method of claim 22, wherein the ratio of the first polymer to the gelation facilitator agent is between about 1:0.03 to about 1:40 by weight.

37. The method of claim 36, wherein the ratio of the first polymer to the gelation facilitator agent is between about 1:0.1 to about 1:20 by weight.

38. The method of claim 37, wherein the ratio of the first polymer to the gelation facilitator agent is between about 1:0.2 to about 1:10 by weight.

39. The method of claim 38, wherein the ratio of the first polymer to the gelation facilitator agent is between about 4:3 to about 3:4 by weight.

40. The method of claim 22, wherein the tablet provides a sustained release of the pharmaceutically active agent for at least about 12 hours.

41. The method of claim 40, wherein the tablet provides a sustained release of the pharmaceutically active agent for up to about 18 hours.

42. The method of claim 23, wherein the pharmaceutically active agent has a solubility of about 0.8 gram/ml in water at a temperature of about 25° C.

43. A method for generating a predetermined sustained release profile of a pharmaceutically active agent, said pharmaceutically active agent is present in at least one particle that is blended with a gel-forming material, which comprises a first polymer, a second polymer, and a gelation facilitator agent, said method comprising adapting different weight percentages of the first polymer, the second polymer, and the gelation facilitator agent in the gel-forming material.

44. The method of claim 43, wherein the pharmaceutically active agent is in contact with a coating material.

45. The method of claim 43, wherein the first polymer is a polyethylene oxide polymer.

46. The method of claim 45, wherein the polyethylene oxide polymer has an average molecular weight of at least about 4×10⁶ Daltons.

47. The method of claim 43, wherein the gelation facilitator agent is polyethylene glycol.

48. The method of claim 47, wherein the polyethylene glycol is a member selected from the group consisting of PEG400, PEG800, PEG1000, PEG1200, PEG1500, PEG2000, PEG4000, PEG6000, PEG8000, PEG10000, and PEG20000.

49. The method of claim 48, wherein the polyethylene glycol is PEG6000 or PEG8000.

50. The method of claim 43, wherein the second polymer is the second polymer consists of one or more polysaccharides selected from the group consisting of locust bean gum, xanthan gum, tragacanth, xylan, arabinogalactan, agar, gellan gum, scleroglucan, guar gum, apricot gum (Prunus armeniaca, L.), alginate, carrageenan, acacia gum, dragon gum, hog gum, talha, dextran, and gum arabic.

51. The method of claim 50, wherein the second polymer consists of xanthan gum.
